(19) United States
(12) Patent Application Publication
(10) Pub. No.: US 2021/0169818 A1
(43) Pub. Date: Jun. 10, 2021

(54) Transdermal Delivery System

(71) Applicant: Xuefei Bai, Allston, MA (US)

(72) Inventor: Xuefei Bai, Allston, MA (US)

(21) Appl. No.: 17/098,328

(22) Filed: Nov. 14, 2020

Related U.S. Application Data
(60) Provisional application No. 62/974,081, filed on Nov. 15, 2019.

(51) Int. Cl. A61K 9/51; C08L 71/02; C08J 3/075

(52) U.S. Cl. CPC A61K 9/5146; A61K 9/5192; A61K 9/5123; C08J 2471/02; C08J 3/075; C08J 2371/02; C08L 71/02

(57) ABSTRACT

The present invention relates to compositions and methods for transdermal delivery of molecules or active ingredients into skin layers underneath Stratum Corneum. In preferred embodiments, the compositions comprise delivery systems providing high transdermal delivery efficiency.

### RELATED APPLICATIONS

This application claims priority benefit of provisional Application Ser. No. 62/974,081 filed Nov. 15, 2019.

### TECHNICAL FIELD

The disclosure relates to nanoparticles and related methods, e.g. methods of making and using nanoparticle agents.

### BACKGROUND

As the largest organ of human's body, skin is the most important barrier shielding us from the environmental substances. For decades, the skincare industry has explored a wide range of nutrients and other active ingredients aiming at the improvement of overall skin health conditions and appearances. However, such active ingredients, especially hydrophilic macromolecules, can hardly permeate the stratum corneum, the first layer of the skin. The key obstacle is incompatibility between the hydrophilic molecules and the lipid matrix filling the interstitial spaces among corneocytes. Efficient and safe delivery of molecules and active ingredients into skin layers underneath stratum corneum has long been considered as one of the most challenging issue in the fields of dermatology as well as cosmetic practice. The current disclosure provides a solution for the long-felt need of transdermal delivery of molecules of various sizes and hydrophilicity.

### SUMMARY OF THE INVENTION

The present disclosure relates to compositions and methods for transdermal delivery of molecules or active ingredients into skin layers underneath stratum corneum. The compositions comprise a novel designed synthetic hydrogel particles with a lipophilic surface capable of efficient delivery of hydrophilic molecules across the stratum corneum. This hydrogel particle carrier was proven to possess a low cytotoxicity to human epidermis. In some aspects, the composition comprises hydrogel particles with a diameter of 10-500 nanometers comprised of a hydrophilic polymer network in a volume of aqueous solution as the core and lipophilic side chains extending out of the volume of aqueous solution as the shell.

This disclosure paves a broad avenue toward effective and economical delivery of active materials in skincare products and transdermal administration of pharmaceutics.

Some aspects of the disclosure relate to a particle with a diameter of 10 to 200 nanometers, comprising a core and lipophilic side chains, wherein the core comprises a volume of aqueous solution and a hydrophilic polymer in the volume of aqueous solution, and wherein the lipophilic side chains extend out of the volume of aqueous solution.

Some aspects of the disclosure relate to a method of making a particle with a diameter of 10 to 200 nanometers, comprising: mixing at least one oil-soluble transfer agent or comonomer in a volume of oil and at least one water-soluble crosslinker in a volume of water; and initiating polymerization with a radical initiator.

Some aspects of the disclosure relate to the method above, wherein the volume of oil further comprises 0-20% by volume nonionic surfactant with a hydrophilic-lipophilic balance of no more than 9 and 0-5% by volume nonionic cosurfactant with an hydrophilic-lipophilic balance of no more than 16.

Some aspects of the disclosure relate to the method above, wherein the volume of oil comprises alpha-olefins, thiols, disulfide, or halide with at least 8 carbons.

Some aspects of the disclosure relate to the method above, wherein the volume of water comprises 20-80% by volume the water-soluble crosslinker.

Some aspects of the disclosure relate to the method above, wherein the radical initiator is a thermal radical initiator, and the polymerization reaction proceeds at a temperature higher than 30° C. for at least 3 hours.

Some aspects of the disclosure relate to the method above, wherein the radical initiator is a redox radical initiator or photo radical initiatioor, and the polymerization reaction proceeds at a temperature not higher than 30° C. for at least 3 hours.

Some aspects of the disclosure relate to the method above, wherein the radical initiator has a concentration of lower than 1% by volume.

Some aspects of the disclosure relate to the particle above, wherein the hydrophilic polymer comprises poly(ethylene glycol) crosslinked by poly(meth)acrylate nodes.

Some aspects of the disclosure relate to the particle above, wherein the lipophilic side chains comprise octadecyl or hexadecyl side chains and are connected to the hydrophilic polymer via a thiolether.

Some aspects of the disclosure relate to the particle above, wherein the hydrophilic polymer comprises poly(ethylene imine), polyacrylamide, poly(N-methylacrylamide) poly(N,N-dimethylacrylamide), poly(N-isopropylacrylamide), poly(N-ethylacrylamide), poly(meth)acrylate, poly(2-hydroxyethyl (meth)acrylate), poly(poly(ethylene glycol) (meth)acrylate), poly(styrenesulfonate), or polysaccharides.

Some aspects of the disclosure relate to the particle above, wherein the lipophilic side chains comprise aliphatic groups containing 6-18 carbons.

Some aspects of the disclosure relate to the particle above, wherein the aliphatic groups containing 6-18 carbons are one or more of 1-hexyl, 1-heptyl, 1-octyl, 1-nonyl, 1-decyl, 1-undecyl, 1-dodecyl, 1-tridecyl, 1-tetradecyl, 1-pentadecyl, 1-heptadecyl, 2-ethylhexyl, 2-hexyldecyl, 7-tridecyl, 9-octadecen-1-yl, 8-heptadecen-1-yl, 9,12-octadecadien-1-yl, or 8,11-heptadecadien-1-yl groups.

Some aspects of the disclosure relate to a composition comprising the particle above, further comprising a pharmaceutically acceptable excipient.

Some aspects of the disclosure relate to the particle above, wherein the percentage that is capable of permeating human epidermis while carrying hyaluronic acid in 15 hours is at least 1%.

Some aspects of the disclosure relate to a method of making a particle, comprising: mixing a mixture containing 1-20% by volume of water-soluble crosslinker, 0.5-20% by volume of a comonomer, 0-20% by volume of a surfactant, 0-5% of a cosurfactant, oil, and water; pre-agitating the mixture; initiating polymerization; demulsifying the mixture; and purifying the mixture.

Some aspects of the disclosure relate to the method above, wherein the comonomer is alpha-olefin.

Some aspects of the disclosure relate to the method above, wherein the surfactant is Brij 93 and the cosurfactant is Brij S10.

Some aspects of the disclosure relate to the particle above, wherein the diameter is 20-50 nanometers, the hydrophilic polymer comprises poly(poly(ethyl glycol) dimethacrylate), and the lipophilic side chains comprise acetylmercapto side chains.

Some aspects of the disclosure relate to the particle above, wherein the diameter is at most 160 nanometers.

### DESCRIPTION OF THE DRAWINGS

FIG. 1 is a conceptual illustration of hydrogel particles with a lipophilic surface prepared by inverse miniemulsion polymerization in the presence of an oil-soluble chain transfer agent or comonomer and its permeation across the stratum corneum.

FIG. 2 shows intensity-weighted hydrodynamic size distribution of the inverse miniemulsion in Example 2.

FIG. 3 shows intensity-weighted hydrodynamic size distribution of the hydrogel particles after the inverse miniemulsion polymerization in Example 2.

FIG. 4 shows intensity-weighted hydrodynamic size distribution of the isolated hydrogel particles in Example 2 after redispersion in mineral oil at a concentration of 10 mg/mL.

FIG. 5 shows cumulative permeation of hydrogel particles across the EpiDerm skin model overtime in comparison to oil-water mixtures (control samples).

FIG. 6 shows relative vialibilty of epiderm cells after permeation experiments of each sample measured by the MTT assay.

FIG. 7 shows intensity-weighted hydrodynamic size distribution of the inverse miniemulsion in Example 1.

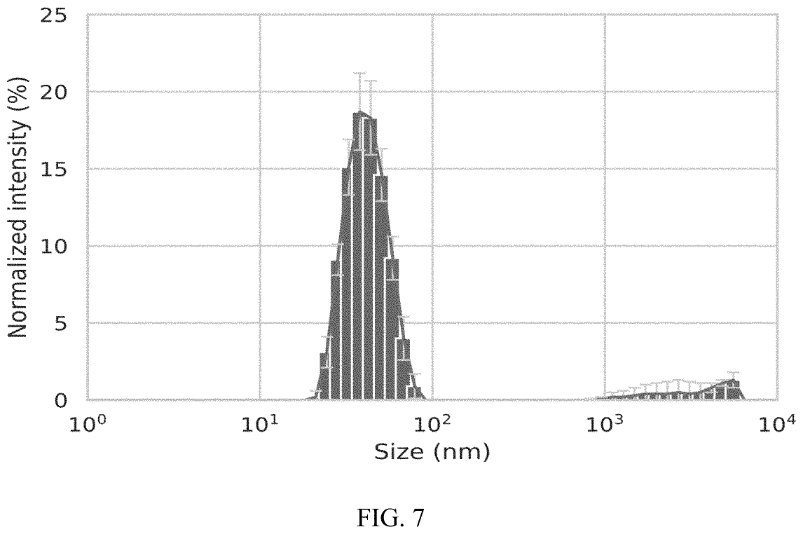

FIG. 8 shows intensity-weighted hydrodynamic size distribution of the hydrogel particles after the inverse miniemulsion polymerization in Example 1.

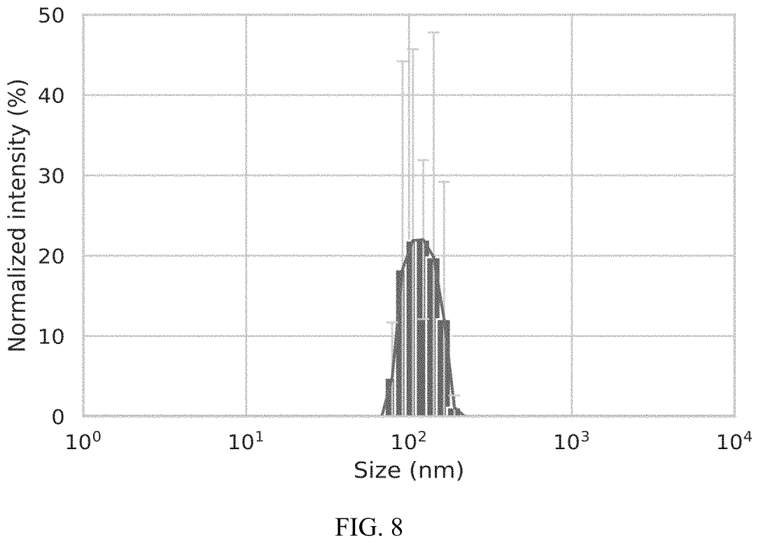

FIG. 9 shows intensity-weighted hydrodynamic size distribution of the isolated hydrogel particles in Example 1 after redispersion in mineral oil at a concentration of 10 mg/mL.

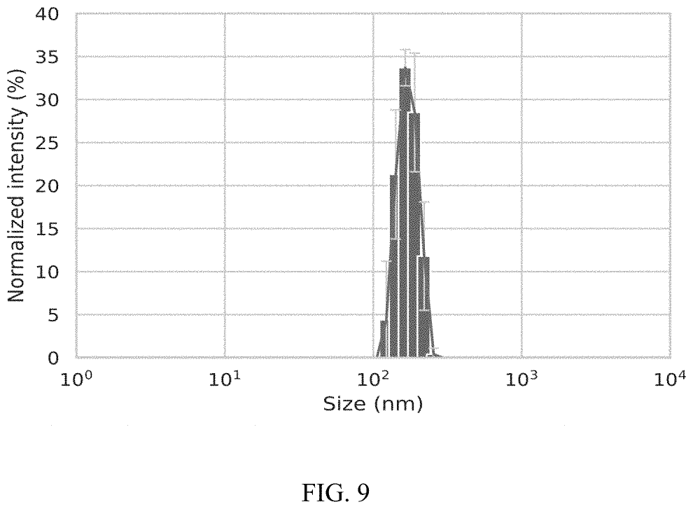

FIG. 10 shows intensity-weighted hydrodynamic size distribution of the inverse miniemulsion in Example 3.

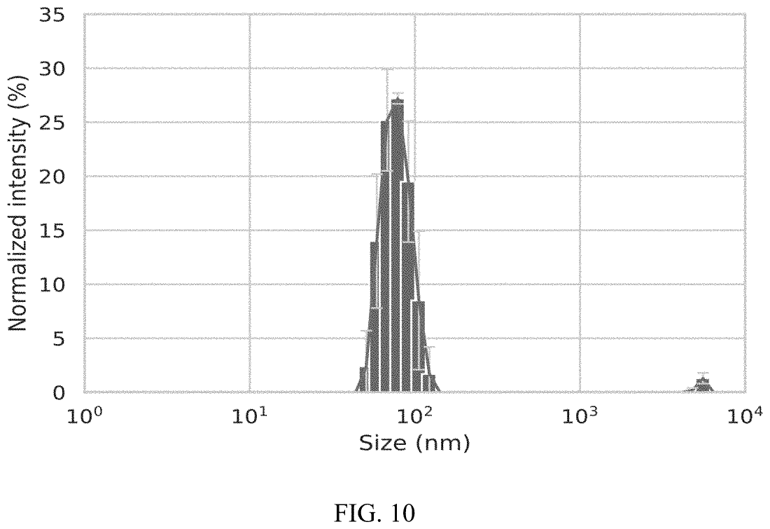

FIG. 11 shows intensity-weighted hydrodynamic size distribution of the hydrogel particles after the inverse miniemulsion polymerization in Example 3.

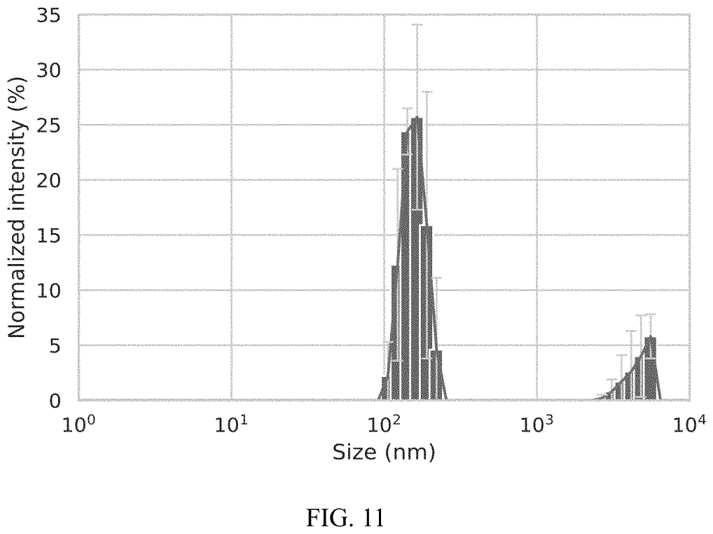

FIG. 12 shows intensity-weighted hydrodynamic size distribution of the isolated hydrogel particles in Example 3 after redispersion in mineral oil at a concentration of 10 mg/mL.

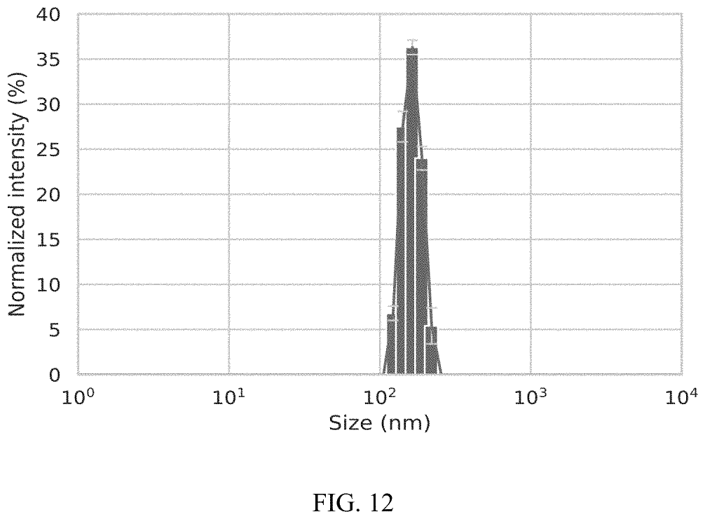

FIG. 13 shows intensity-weighted hydrodynamic size distribution of the isolated hydrogel particles in Example 1 after dehydration and redispersion in mineral oil at a concentration of 10 mg/mL.

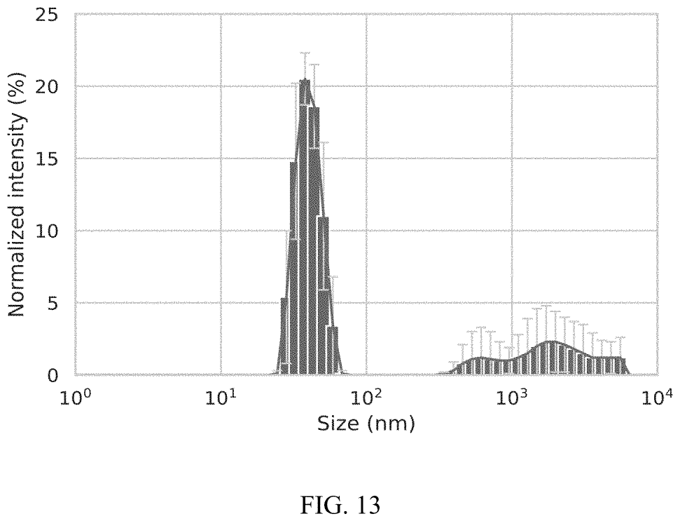

### DETAILED DESCRIPTION

### I. Definitions

To facilitate an understanding of the present disclosure, a number of terms and phrases are defined below:

The term “radical initiator” refers to a compound or a mixture of compounds that can produce radical species and initiate the radical polymerization of a vinyl-based monomer with an external stimuli, including elevated temperature and electromagnetic radiation, or via a redox reaction, and can be selected from diazo compounds, peroxides, persulfates, N-alkoxyamines, phenone derivatives, combinations of peroxides/persulfates and reducing agents such as amines or low-valency metal salts, combinations of dithioesters and metal complexes, or combinations of and alcohols and high-valency metal salts, or -combinations of alkyl halides and metal salts and complexes. Examples of radical initiators include, but are not limited to, 2,2′-azobis(isobutyronitrile), 2,2′-azobis(2-cyanovaleric acid), benzoyl peroxide, lauroyl peroxide, potassium persulfate, ammonium persulfate, 2,2,6,6-tetramethyl-1-(1-phenylethoxy)piperidine, benzophenone, 2,2-dimethoxy-1,2-diphenylethan-1-one, benzoyl peroxide with N-dimethylaniline, ammonium persulfate with iron(II) sulfate, benzyl alcohol with cerium(IV) sulfate, 4-cyano-4-(phenylcarbonothioylthio)pentanoic acid with tris[2-phenylpyridinato-C²,N]iridium(III), and 2-hydroxyethyl 2-bromo-2-methylpropionate with [N,N,N′,N″,N″-pentamethyldiethylenetriamine]copper(I) bromide.

The term “water soluble crosslinker” refers to a telechelic oligomer or polymer with at least two acrylate, methacrylate, acrylamide, methacrylamide, or allyl units. Examples include, but are not limited to, poly(ethylene glycol) diacrylate, poly(ethylene glycol) dimethacrylate; poly(ethylene imide) diacrylamide and poly(ethylene glycol) diallyl ether.

The term “oil” refers to any combination of one or more nonpolar substances, which is a liquid with viscosity larger than water, and is immiscible with water while miscible with other oils. Examples include, but are not limited to, pure or a mixture of mineral oil, liquid paraffin, poly(alpha-olefin), alkanes with at least 8 carbons, olefins with at least 8 carbons, fatty acid esters, and other hydrocarbons.

The terms “surfactant” and “cosurfactant” refer to substances that spontaneously assemble at an oil-water interface to reduce the interfacial energy. Examples include, but are not limited to, Brij 93, Brij 58, Brij S10, Brij S20, Brij 5100, Brij 020, Brij C10, Brij L4, Span 20, Span 40, Span 60, Span 65, Span 80, Span 85, Tween 20, Tween 21, Tween 40, Tween 60, Tween 65, Tween 80, and Tween 85.

The term “hydrophilic-lipophilic balance” refers to a measure of the degree to which a surfactant is hydrophilic or lipophilic as defined by Griffin in 1949¹ and 1954². ¹Griffin, William C (1949), “Classification of Surface-Active Agents by ‘HLB’”, Journal of the Society of Cosmetic Chemists, 1 (5): 311-26²Griffin, William C (1954), “Calculation of HLB Values of Non-Ionic Surfactants”, Journal of the Society of Cosmetic Chemists, 5 (4): 249-56

The term “transfer agent” refers to a substance that reacts with a radical polymerization chain end resulting a fragment of the substance to incorporate to the chain end and another radical fragment to initiate a new polymer chain. Examples include, but are not limited to, 1-octadecanethiol, 1-hexadecanethiol, 1,1′-hexadecyl disulfide, 1,10-diiododecane, and 1,8-diiodooctane.

The term “comonomer” refers to a substance that reacts with a radical polymerization chain end resulting a complete incorporation of the substance to the chain end which can continue with the polymerization. Examples include, but are not limited to, alfa-olefins such as 1-octadecene, 1-hexadecene, or 1-dodecene and vinyl ethers such as octadecyl vinyl ether, hexadecyl vinyl ether, dodecyl vinyl ether, and hexyl vinyl ether as an oil-soluble comonomer and sodium acrylate, sodium 4-styrenesulfonate, sodium methacrylate, acrylamide, N,N-dimethylacrylamide, and sodium 2-acrylamido-2-methylpropanesulfonate.

The term “homogenizer” refers to a device that homogenizes a blend of materials via a mechanical disruption. Examples of the mechanical disruption include, but are not limited to, ultrasound and rotational shear stress.

The term “miniemulsion polymerization” refers to a polymerization of an emulsion of monomer in which all of the polymerization occurs within the preexisting monomer particles with diameters in the range from approximately 50 nanometers to 1 micrometer as defined by the International Union of Pure and Applied Chemistry (IUPAC).³ ³Slomkowski, Stanislaw; Alemán, José V.; Gilbert, Robert G.; Hess, Michael; Horie, Kazuyuki; Jones, Richard G.; Kubisa, Przemyslaw; Meisel, Ingrid; Mormann, Werner; Penczek, Stanislaw; Stepto, Robert F. T. (2011). “Terminology of polymers and polymerization processes in dispersed systems (IUPAC Recommendations 2011)”. Pure and Applied Chemistry. 83 (12): 2229-2259

The term “diameter” refers to the longest chord of a particle.

The term “demulsifier” refers to a substance or a mixture capable of destabilization of an emulsion. Examples include but not limited to acetone, ethanol, methanol, isopropanol, and sodium chloride.

The term “cumulative permeation” refers to the percentage of loaded hydrogel particles permeated across the skin model overtime calculated from the sum of concentrations of hydrogel nanoparticles as quantified by the rhodamine B tracer at each timepoint and those at all previous timepoints.

### II. The Invention

The present disclosure is directed to compositions and methods for transdermal delivery of molecules or active ingredients into skin layers underneath stratum corneum. The compositions may be hydrogel particles with a diameter of 10-500 nanometers comprised of a hydrophilic polymer network in a volume of aqueous solution as the core and lipophilic side chains extending out of the volume of aqueous solution as the shell.

In some aspects, a hydrophilic polymer network is comprised of poly(ethylene glycol) (molecular weight 500˜2000) chemically crosslinked by poly(meth)acrylate nodes.

In some aspects, lipophilic octadecyl or hexadecyl side chains are connected to crosslinking points of hydrophilic polymer network via a thiolether.

In some aspects, a hydrophilic polymer network is comprised of water soluble polymer skeletons including, poly(ethylene imine), polyacrylamide, poly(N-methylacrylamide) poly(N,N-dimethylacrylamide), poly(N-isopropylacrylamide), poly(N-ethylacrylamide), poly(meth)acrylate, poly(2-hydroxyethyl (meth)acrylate), poly(poly(ethylene glycol) (meth)acrylate), poly(styrenesulfonate), polysaccharides, etc.

In some aspects, crosslinking points comprised of covalent multifunctional structures, including silsesquioxanes, pentaerythritol esters, tertiary amines, glycerol ethers, metal complexes, etc. or multifunctional noncovalent structures, including polyelectrolyte coacervates, hydrogen bondings, π-π stackings, etc.

In some aspects, lipophilic side chains comprised of linear or branched, saturated or unsaturated aliphatic groups containing 6-18 carbons, including 1-hexyl, 1-heptyl, 1-octyl, 1-nonyl, 1-decyl, 1-undecyl, 1-dodecyl, 1-tridecyl, 1-tetradecyl, 1-pentadecyl, 1-heptadecyl, 2-ethylhexyl, 2-hexyldecyl, 7-tridecyl, 9-octadecen-1-yl, 8-heptadecen-1-yl, 9,12-octadecadien-1-yl, 8,11-heptadecadien-1-yl, etc., are connected to the crosslinking points of the hydrophilic polymer network via a thiolether, an amide, an ester, or a carbon-carbon bond.

### A. General Preparation of Hydrogel Particles

In some aspects, hydrogel particles are prepared in an inverse miniemulsion polymerization comprised of at least one nonionic surfactant, at least one oil-soluble transfer agent or comonomer dissolved in an oil and at least one water-soluble crosslinker with or without at least one water-soluble comonomer dissolved in water, initiated by a radical initiator. The resulting hydrogel particles are isolated by a least one demulsifier. The residual oil and surfactant(s) are removed by solvent washes.

In some aspects, in an oil, 0-20% of a nonionic surfactant with a hydrophilic-lipophilic balance (HLB) no more than 9 and 0-5% a nonionic cosurfactant with an HLB no more than 16 is dissolved. The oil may comprise pure or a mixture of mineral oil, liquid paraffin, poly(alpha-olefin), alkanes with at least 8 carbons, olefins with at least 8 carbons, or other hydrocarbons. Examples of the surfactants and the cosurfactants include but are not limited to Brij 93, Brij 58, Brij S10, Brij S20, Brij S100, Brij 020, Brij C10, Brij L4, Span 20, Span 40, Span 60, Span 65, Span 80, Span 85, Tween 20, Tween 21, Tween 40, Tween 60, Tween 65, Tween 80 and Tween 85.

In some aspects, at least one oil-soluble transfer agent or comonomer is mixed with the aforementioned mixture to a final concentration of 0.5-20%. The transfer agent or comonomer may comprise at least one of alpha-olefins, thiols, disulfide, or halide with at least 8 carbons. Examples of transfer agents or comonomers include but are not limited to 1-octadecene, 1-hexadecene, 1-dodecene, 1-octadecanethiol, 1-hexadecanethiol, 1,1′-hexadecyl disulfide, 1,10-diiododecane and 1,8-diiodooctane.

In some aspects, at least one water-soluble crosslinker with or without a water-soluble comonomer is dissolved in water at a concentration of 20-80%, making an aqueous solution. The aqueous solution is mixed with the aforementioned oil solution. In some aspects, the aqueous solution comprises a concentration of 5-15%. The mixture is homogenized extensively.

In some aspects, the mixture is mixed with a homogenizer.

In some aspects, one thermal, redox, or photo radical initiator is introduced to the mixture at a concentration of <1% before or after the mixture is degassed.

In some aspects, the reaction proceeds at an elevated temperature if a thermal initiator is used or at room temperature if a redox or photo initiator is used for at least 3 hours to give the hydrogel particles with a lipophilic surface.

In some aspects, the synthesized hydrogel particles are isolated by demulsification with a demulsifier.

In some aspects, the oil and surfactant residues on the hydrogel particles are washed away with a nonpolar solvent such as, but not limited to, hexanes, pentanes, heptanes, cyclohexane, benzene, toluene, xylenes, chlorobenzene, dichloromethane, chloroform, carbon tetrachloride, ethyl actetate and diethyl ether.

In some aspects, the solvent residue is allowed to evaporate at an ambient condition.

In some aspects, a mixture containing poly(ethyl glycol) dimethacrylate 750, mineral oil, acetyl mercaptan, Brij 93, Brij S10, ammonium persulfate, water on a weight ratio of 60:320:35:30:10:1:60 is mixed in reaction flask charged with a cross-shaped magnetic stir bar. The mixture is pre-agitated with a shear-force homogenizer to form an inverse microemulsion at 0° C. Then a nitrogen flow is purged through the microemulsion to remove oxygen. The polymerization is initiated by heating the reaction mixture to 50° C. The mixture was stirred for 20 hours at 50° C. The resulting mixture is demulsified by adding an excess of acetone and purified by washing with hexanes. The hydrogel particles with diameters of 20-50 nanometers thus yielded is comprised of crosslinked poly(poly(ethyl glycol) dimethacrylate) network swollen by an aqueous solution and cetylmercapto side chains.

### B. Examples

The following examples are provided in order to demonstrate and further illustrate certain aspects of the present disclosure and are not to be construed as limiting the scope thereof.

As described herein, in each stage of the synthesis and tests, the samples were characterized by dynamic light scattering to monitor the change in sizes of the hydrogel particles and their dispersibility in an oil. The hydrodynamic size of the inverse miniemulsion or hydrogel particles were analyzed using a Malvern Zetasizer Nano S particle size analyzer.

As described herein, the hydrogel particles were traced using rhodamine B (RhB). In one aspect, for every gram of the hydrogel particles 50 μL of 2.0% RhB (Alfa Aesar) in milliQ water solution was added as a tracer. A linear calibration curve was established for RhB concentrations of 1000, 500, 200, 100, 50, 20, 10, 5, 2, 1 ng/mL (r2>0.998) by fluorescence readouts using a plate reader (Promega GM3500) at 520 nm excitation and 580-640 nm emission. The same parameters were used to establish the concentration relationship between RhB and hydrogel particles.

As described herein, EpiDerm (MatTek Corporation, Ashland, Mass.) Skin Model EPI-212-X was used for permeation studies. The EpiDerm tissues were placed in fresh media and incubated at 37° C., RH=5% incubator overnight. Each tissue insert was then placed in the MatTek Permeation Device (MPD), a reusable permeation device which directly accepts the EPI-212-X tissues. After stabilizing tissue inserts in MPDS with 5 mL fresh media EPI-100-LLMM-X-PRF (MatTek Corporation, Ashland, Mass.) for one hour, 0.5 mL of each test material containing 10 mg/mL hydrogel particles or 10 aqueous solution of RhB dispersed in mineral oil was added, with triplicates. 1 mg/mL RhB were added in the control samples while 1 mg/mL hydrolyzed hyaluronic acid was also added to the HA controls. Tissues were incubated at 37° C., RH=5% incubator. Samples of receptor fluid were taken completely at various time intervals and were assessed for permeant concentration. The receptor solution was replaced with fresh mead at each time point. The concentration of permeating particles was measured using aforementioned method.

As described herein, the MTT solution was added at 24 h after epiderm tissues were exposed to samples. Approximately 1 hour prior to the end of the dosing period, the MTT solution was prepared using the MatTek MTT toxicology kit (Part #MTT-100). 15 min before each dosing period is complete, a 24-well plate with MTT solution was prepared. 300 μL of the MTT solution was added into the appropriate number of wells of the 24-well plate to accommodate all the inserts. After exposure of the EpiDerm samples to the test materials was complete, any liquid residue atop the EpiDerm tissues was decanted. Each insert was removed individually and gently rinsed twice with PBS. Excess liquid was shaken off prior to placing the EpiDerm sample in the MTT-containing 24-well plate. The EpiDerm samples in the 24-well plate was placed in the incubator for 3 hours. Then, each insert was removed individually and gently blotted with a KimWipe. Finally, the inserts were placed into the 24-well extraction plate. The cell culture inserts were immersed with 2.0 ml of the extractant solution per well to completely cover the EpiDerm sample. The extraction plate was placed with its lid into a Ziplock bag. The extraction was allowed to proceed overnight without shaking at room temperature in the dark. Then, the liquid within each insert was decanted back into their corresponding original wells. The inserts were discarded. The extractant solution were thoroughly mixed and transferred in 200 μL aliquots with triplicates. The optical density of the extracted samples was determined at 570 nm using 200 μl of extractant as a blank and the viability was determined using the following equation.

% viability=100×[OD(sample)/OD(negative control)]

### Example 1

3.7 g of Brij 93 (Sigma-Aldrich), 0.3 g of Brij S10 (Sigma-Aldrich), and 4 mL of 1-octadecene (Alfa Aesar) were dissolved in 40 mL of mineral oil by stirring in a 100-mL round bottom flask with a cross-shaped magnetic stir bar. 3 grams of poly(ethylene glycol) dimethacrylate (PEGDMA) 750 (Sigma-Aldrich) was dissolved in 3 mL of milliQ water. The aqueous solution was added into the oil solution while stirring. The mixture is homogenized using an ultrasonication probe for 10 min. 0.1 g of ammonium persulfate (Alfa Aesar) was added into the reaction mixture while stirring. The reaction flask was sealed and bubbled with nitrogen for 30 min at a rate of 1-3 bubbles per second measured by a mineral oil bubbler. 50 μL of N,N-dimethylaniline (Alfa Aesar) was injected using micro syringe into the flask after bubbling. The reaction was stirred at room temperature for 3 hours. The reaction was quenched by exposure to the air. 15 mL of acetone (Alfa Aesar) was added to demulsify the mixture. The hydrogel was precipitated by centrifuge at 3000 rpm for 3 minutes. The mixture was washed twice by redisperse in 20 mL of hexanes (Alfa Aesar). The hydrogel was let dry in open air and stored at 4° C. after the hexanes thoroughly evaporated.

### Example 2

3.7 g of Brij 93, 0.3 g of Brij S10, and 4 mL of 1-octadecene were dissolved in 40 mL of mineral oil by stirring in a 100-mL round bottom flask with a cross-shaped magnetic stir bar. 3 grams of poly(ethylene glycol) dimethacrylate (PEGDMA) 750 was dissolved in 3 mL of milliQ water. The aqueous solution was added into the oil solution while stirring. The mixture is homogenized using an ultrasonication probe for 10 min. 0.1 g of ammonium persulfate was added into the reaction mixture while stirring. The reaction flask was sealed and bubbled with nitrogen for 30 min at a rate of 1-3 bubbles per second measured by a mineral oil bubbler. The reaction was stirred at 50° C. for 20 hours. The reaction was quenched by exposure to the air. 15 mL of acetone was added to demulsify the mixture. The hydrogel was precipitated by centrifuge at 3000 rpm for 3 minutes. The mixture was washed twice by redisperse in 20 mL of hexanes. The hydrogel was let dry in open air and stored at 4° C. after the hexanes thoroughly evaporated.

### Example 3

3.7 g of Brij 93, 0.3 g of Brij S10, and 4 mL of 1-octadecene were dissolved in 40 mL of mineral oil by stirring in a 100-mL round bottom flask with a cross-shaped magnetic stir bar. 3 grams of poly(ethylene glycol) dimethacrylate (PEGDMA) 750 and 3 mg of briefly hydrolyzed hyaluronic acid (Alfa Aesar) were dissolved in 3 mL of milliQ water. The aqueous solution was added into the oil solution while stirring. The mixture is homogenized using an ultrasonication probe for 10 min. 0.1 g of ammonium persulfate was added into the reaction mixture while stirring. The reaction flask was sealed and bubbled with nitrogen for 30 min at a rate of 1-3 bubbles per second measured by a mineral oil bubbler. The reaction was stirred at 50° C. for 20 hours. The reaction was quenched by exposure to the air. 15 mL of acetone was added to demulsify the mixture. The hydrogel was precipitated by centrifuge at 3000 rpm for 3 minutes. The mixture was washed twice by redisperse in 20 mL of hexanes. The hydrogel was let dry in open air and stored at 4° C. after the hexanes thoroughly evaporated.

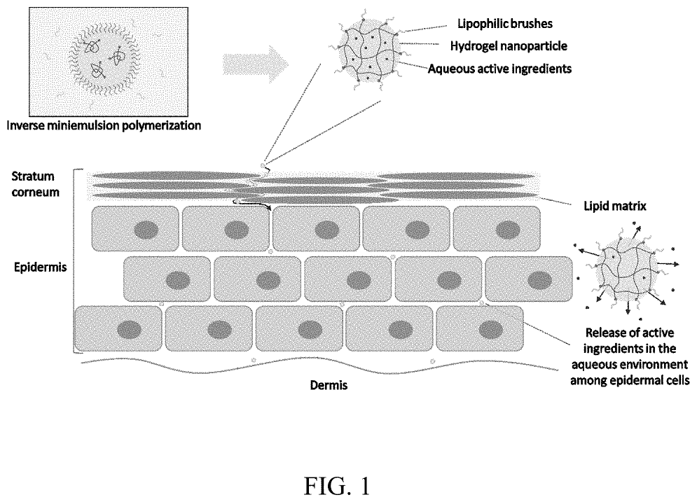

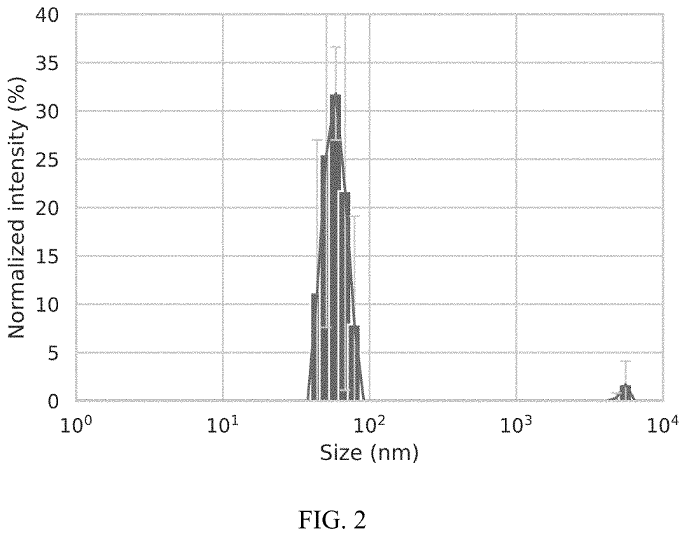

In some aspects, preparation of the hydrogel particles with a lipophilic surface was based on inverse miniemulsion polymerization (FIG. 1). The oligomeric/polymeric crosslinkers were trapped inside aqueous droplets of ca. 100 nm stabilized by surfactants with a low HLB in the oil medium (FIG. 2). Hydrophilic macromolecular active ingredients such as hyaluronic acid or hydrolyzed collagen can be loaded prior to polymerization while small molecules such as ascorbic acid or nicotinamide can be loaded before or after polymerization. During the radical polymerization, the crosslinkers establishes a network loosely restrained by the size of the aqueous droplets. The propagating radicals encounter the oil soluble comonomers or transfer agents at the oil-water interface. In the case of alpha-olefins, due to its inability of homopolymerization, the radical cannot propagate into the oil phase. Instead, it incorporates at the crosslinking points of the hydrogel network as individual units. These lipophilic chains cover the surface of the hydrogel particles boosting their dispersibility and stability in an oily medium.

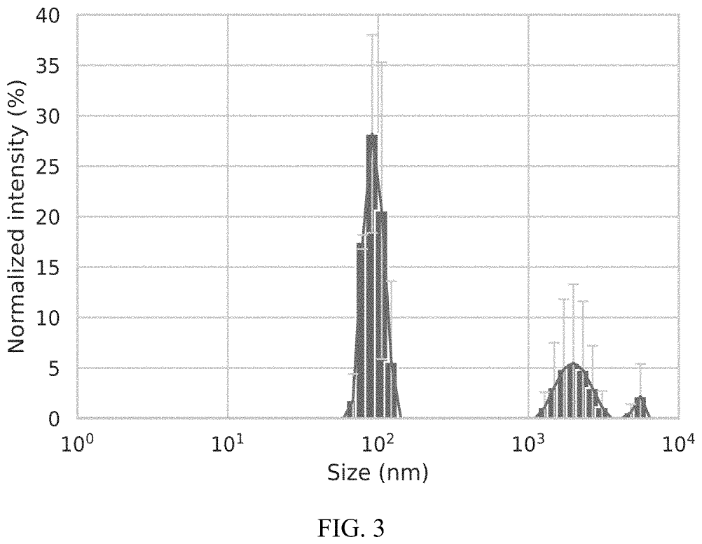

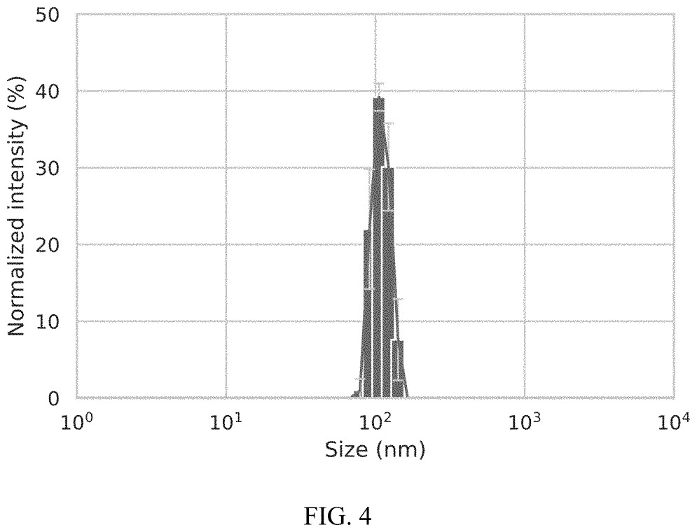

While majority of the hydrogel particles retained the initial size of the aqueous droplets, a small fraction of them aggregated due to thermal destabilization of the miniemulsion after the polymerization (FIG. 3). These aggregates could be removed during isolation. The resulting hydrogel particles are stable as a semi-solid or solid, which can be redispersed in an oily medium in a size of ca. 100 nm (FIG. 4).

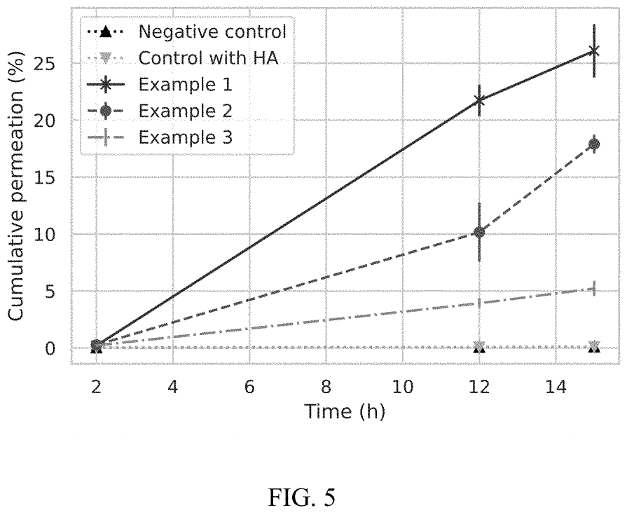

When hydrophilic molecules are deposited directly on the skin, they tend to aggregate into much larger sizes than the gap between corneocytes, essentially obstructing intake of these active ingredients. However, when these same molecules are loaded inside the hydrogel particles with a lipophilic surface as carriers. The lipophilic surface compatibilizes the hydrogel particles with the lipid among corneocytes while the hydrophilic active materials stay solvated by water inside the hydrogel particles. As demonstrated by the permeation experiment using EpiDerm skin models, up to 25% of hydrogel particles carried the RhB dye across the epidermis within 15 hours. Meanwhile, only a trace of RhB solution dispersed in mineral oil could cross the same skin model (FIG. 5).

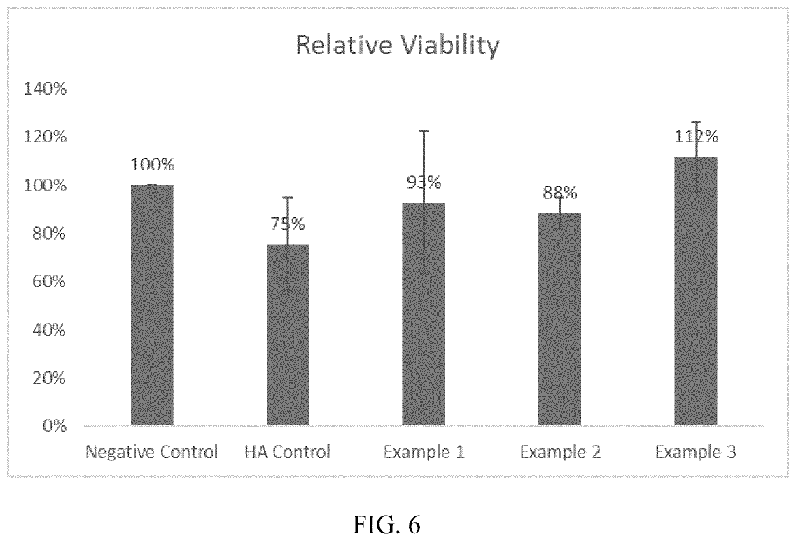

Moreover, after 24-hour exposure to the hydrogel particles, the epidermal cells in the skin models remained comparable viability to the cells exposed to a negative control indicating a non-irritant nature of these hydrogel particles (FIG. 6).

### III. Formulations

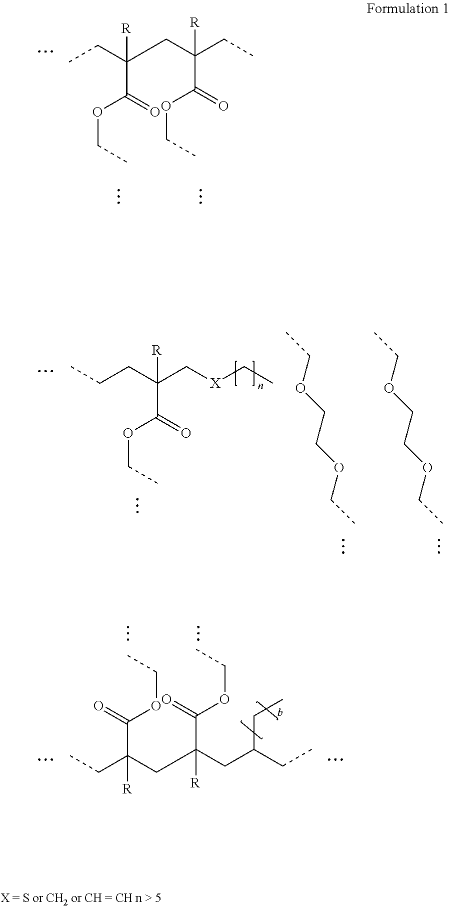

Formulation 1 represents an exemplary structure of the hydrogel particle carrier comprised of a poly(ethylene glycol) skeleton crosslinked by poly(meth)acrylates with lipophilic side chains attached via a thiolether bond. R═H or Me; n>5. Dashed lines indicate an indefinite extension of the repeating structure moieties.

We claim:

1. A particle with a diameter of 10 to 200 nanometers, comprising a core and lipophilic side chains, wherein the core comprises a volume of aqueous solution and a hydrophilic polymer in the volume of aqueous solution, and wherein the lipophilic side chains extend out of the volume of aqueous solution.

2. A method of making a particle with a diameter of 10 to 200 nanometers, comprising:
mixing at least one oil-soluble transfer agent or comonomer in a volume of oil and at least one water-soluble crosslinker in a volume of water; and
initiating polymerization with a radical initiator.

3. The method of claim 2, wherein the volume of oil further comprises 0-20% by volume nonionic surfactant with a hydrophilic-lipophilic balance of no more than 9 and 0-5% by volume nonionic cosurfactant with an hydrophilic-lipophilic balance of no more than 16.

4. The method of claim 2, wherein the volume of oil comprises alpha-olefins, thiols, disulfide, or halide with at least 8 carbons.

5. The method of claim 2, wherein the volume of water comprises 20-80% by volume the water-soluble crosslinker.

6. The method of claim 2, wherein the radical initiator is a thermal radical initiator, and the polymerization reaction proceeds at a temperature higher than 30° C. for at least 3 hours.

7. The method of claim 2, wherein the radical initiator is a redox radical initiator or photo radical initiatioor, and the polymerization reaction proceeds at a temperature not higher than 30° C. for at least 3 hours.

8. The method of claim 2, wherein the radical initiator has a concentration of lower than 1% by volume.

9. The particle of claim 1, wherein the hydrophilic polymer comprises poly(ethylene glycol) crosslinked by poly(meth)acrylate nodes.

10. The particle of claim 1, wherein the lipophilic side chains comprise octadecyl or hexadecyl side chains and are connected to the hydrophilic polymer via a thiolether.

11. The particle of claim 1, wherein the hydrophilic polymer comprises poly(ethylene imine), polyacrylamide, poly(N-methylacrylamide) poly(N,N-dimethylacrylamide), poly(N-isopropylacrylamide), poly(N-ethylacrylamide), poly(meth)acrylate, poly(2-hydroxyethyl (meth)acrylate), poly(poly(ethylene glycol) (meth)acrylate), poly(styrenesulfonate), or polysaccharides.

12. The particle of claim 1, wherein the lipophilic side chains comprise aliphatic groups containing 6-18 carbons.

13. The particle of claim 12, wherein the aliphatic groups containing 6-18 carbons are one or more of 1-hexyl, 1-heptyl, 1-octyl, 1-nonyl, 1-decyl, 1-undecyl, 1-dodecyl, 1-tridecyl, 1-tetradecyl, 1-pentadecyl, 1-heptadecyl, 2-ethylhexyl, 2-hexyldecyl, 7-tridecyl, 9-octadecen-1-yl, 8-heptadecen-1-yl, 9,12-octadecadien-1-yl, or 8,11-heptadecadien-1-yl groups.

14. A composition comprising the particle of claim 1, further comprising a pharmaceutically acceptable excipient.

15. The particle of claim 1, wherein the percentage that is capable of permeating human epidermis while carrying hyaluronic acid in 15 hours is at least 1%.

16. A method of making a particle, comprising:
mixing a mixture containing 1-20% by volume of water-soluble crosslinker, 0.5-20% by volume of a comonomer, 0-20% by volume of a surfactant, 0-5% of a cosurfactant, oil, and water;
pre-agitating the mixture;
initiating polymerization;
demulsifying the mixture; and
purifying the mixture.

17. The method of claim 16, wherein the comonomer is alpha-olefin.

18. The method of claim 16, wherein the surfactant is Brij 93 and the cosurfactant is Brij S10.

19. The particle of claim 1, wherein the diameter is 20-50 nanometers, the hydrophilic polymer comprises poly(poly(ethyl glycol) dimethacrylate), and the lipophilic side chains comprise acetylmercapto side chains.

20. The particle of claim 1, wherein the diameter is at most 160 nanometers.
